(19) United States
(12) Patent Application Publication
(10) Pub. No.: US 2015/0038362 A1
(43) Pub. Date: Feb. 5, 2015

(54) High-Throughput Method For Sialic Acid Quantitation

(71) Applicant: Biogen Idec MA Inc., Cambridge, MA (US)

(72) Inventors: Lam Raga Anggara Markely, Cambridge, MA (US); Shashi Prajapati, Acton, MA (US)

(21) Appl. No.: 14/380,983

(22) PCT Filed: Feb. 27, 2013
(86) PCT No.: PCT/US2013/028017

§ 371 Date: Aug. 26, 2014

Related U.S. Application Data
(60) Provisional application No. 61/603,529, filed on Feb. 27, 2012.

(51) Int. Cl. C12Q 1/34; G01N 33/68

(52) U.S. Cl. CPC C12Q 1/34; G01N 33/6854; G01N 2333/924; G01N 2440/38
USPC 506/9; 435/7.1; 506/39; 435/287.9

(57) ABSTRACT

The present invention relates to a method for specifically measuring sialylation of a biomolecule of interest without the interference of other biomolecules present in the sample.

### BACKGROUND OF THE INVENTION

1. Field of the Invention

The present invention relates to a method for specifically measuring sialylation of a biomolecule of interest without the interference from other biomolecules present in the sample.

2. Background Art

Sialic acid is important for therapeutic proteins because it can improve serum half-life (F. A. Ngantung et al. Biotechnol. Bioeng. 95:106-119 (2006)), biological activity (Y. Kaneko et al. Science 313:670-673 (2006)), solubility (A. M. Sinclair et al. J. Pharm. Sci. 94:1626-1635 (2005)), resistance to thermal denaturation, and protease attack (E. Tsuda et al. Eur. J. Biochem. 188:405-411 (1990); E. Goldwasser et al. J. Biol. Chem. 249:4202-4206 (1974)) of the proteins. In order to ensure that the sialylation of proteins is consistent and optimum, a high-throughput method (HTM) for quantifying glycoprotein sialylation is required in bioprocesses. During cell line development, for instance, thousands of clones need to be analyzed to select the best clone. This task is further complicated by intraclonal variability and production instability observed in previous studies (W. Pilbrough et al. PLoS One 4 (2009); M. Kim et al. Biotechnol. Bioeng. 108:2434-2446 (2011); and K. R. Love et al. Biotechnol. Bioengin. 106:319-325 (2010)). Moreover, many bioprocess parameters can affect the sialylation (L. Santell et al. Biochem. Biophys. Res. Comm. 258:132-137 (1999); M. Gawlitzek et al. Biotechnol. Bioengin. 68:637-646 (2000); M. Gawlitzek et al. Biotechnol. Bioengin. 103:1164-1175 (2009); S. W. Harcum, Protein Glycosylation. in: S. S. Ozturk, and W.-S. Hu, (Eds.), Cell culture technology for pharmaceutical and cell-based therapies, Taylor & Francis, New York, 2006, pp. 113-154; P. Hossler et al. Glycobiology 19:936-949 (2009); L. R. A. Markely, Chem. Engineering, Massachusetts Institute of Technology, Cambridge, 2011; D. C. F. Wong et al. Biotechnol. Bioengin. 89:164-177 (2005); D. C. F. Wong et al. Biotechnol. Bioengin. 107:516-528 (2010); N. S. C. Wong et al. Biotechnol. Bioengin. 107:321-336 (2010); and E. K. Read et al. Biotechnol. Bioengin. 105:276-284 (2010)). Thus, analyses of the effects of numerous culture conditions on the sialylation are crucial to identify optimum process conditions and consistently control the product qualities.

Ideally, the HTM should be able to analyze hundreds of samples in parallel, require only small volume of crude culture supernatant, and finish the analysis in a few minutes (E. K. Read et al. (2010)). In addition, it has to be specific, sensitive, precise, and accurate compared to some colorimetric, chromatographic, enzymatic, and fluorescence methods that have been widely used for analyzing recombinant proteins (M. Gawlitzek et al. (2000); K. P. Gopaul et al. Clin. Biochem. 39:667-681 (2006); N. S. C. Wong et al. Biotechnol. Bioengin. 93:1005-1016 (2006); and K. R. Anumula, Anal. Biochem. 230:24-30 (1995)).

The ability to analyze small volume of samples is critical for cell clone screening because the culture volumes during primary and secondary screenings typically range from about 100 μL to about 3 mL. This task also requires an HTM that is not interfered by sialylated host cell proteins (HCP) and other biomolecules secreted by the cells. This criterion is important because product titers are usually low at this screening stage, and the concentration of sialic acid from HCP and other impurities can be much higher than the product. This feature is also important for bioprocess development because changes in process conditions may alter the sialylation of HCP and thus, make the HTM inaccurate.

In a previous study, Park et al. adapted a microengraving method (J. C. Love et al. Nature Biotechnol. 24:703-707 (2006)) to develop an HTM for quantifying sialylation of glycoproteins secreted in single cell cultures (S. Park et al. Anal. Chem. 82:5830-5837 (2010)). Their HTM can analyze many crude culture samples in parallel in one to two days. In another study, an HTM for quantifying glycoprotein sialylation was developed (L. R. A. Markely et al. Anal. Biochem. 407:128-133 (2010)). This HTM can rapidly (15 minutes) analyze many crude culture samples in parallel. The HTM has been validated for monitoring sialylation of interferon-γ (IFN-γ) produced in Chinese Hamster Ovary (CHO) cell cultures. Although the HTM was free from interference by chemicals, such as glucose and pyruvate, in culture samples, it could not distinguish sialic acid of the product from sialic acid of other biomolecules in the samples. Thus, the HTM could not specifically measure sialic acid of IFN-γ, and used overall sialylation of all the proteins in culture samples to estimate IFN-γ sialylation.

Therefore, there exists a need in the art for rapid, high-throughput assays to specifically measure sialylation of a biomolecule of interest, for instance protein, peptide, and lipid, without the interference of other molecules present in the sample.

### BRIEF SUMMARY OF THE INVENTION

The present invention is directed to a method of detecting sialylation of a biomolecule of interest in a sample comprising: (a) purifying the biomolecule of interest by contacting the sample with an agent that binds the biomolecule of interest; (b) denaturing the biomolecule of interest by incubating the sample with a surfactant; (c) contacting the denatured sample with an agent capable of removing terminal sialic acid residues from the biomolecule of interest, thereby generating free sialic acid residues; (d) labeling the free sialic acid residues with a detectable label; (e) detecting the labeled sialic acid residues, thereby detecting sialylation of the biomolecule of interest. In one embodiment, the method further comprises quantifying the level of sialylation of the biomolecule of interest.

In one embodiment, the sample comprises a cell culture supernatant. In another embodiment, the sample comprises a clinical sample.

In one embodiment, the sample is located in a multi-well vessel. In another embodiment, the multi-well vessel comprises up to 96 wells. In another embodiment, the multi-well vessel comprises greater than 96 wells. In another embodiment, the multi-well vessel comprises 384 wells. In another embodiment, the multi-well vessel comprises 1536 wells.

In one embodiment, the agent that binds the biomolecule of interest is protein A. In another embodiment, the surfactant is sodium 3-[(2-methyl-2-undecyl-1,3-dioxolan-4-yl)methoxy]-1-propanesulfonate.

In one embodiment, the denaturation occurs between about 50° C. and about 80° C. In another embodiment, the denaturation occurs at between about 60° C. and about 70° C. In another embodiment, the denaturation occurs at about 60° C.

In one embodiment, the terminal sialic acid residues are removed by contacting the denatured sample with an enzyme. In one embodiment, the enzyme is sialidase.

In one embodiment, the labeling agent is malonitrile. In another embodiment, the labeling is performed at a temperature of between about 60° C. and about 100° C. In another embodiment, the labeling is performed at 80° C.

In one embodiment, the free sialic acid is detected using a spectrophotometer or a fluorometer. In another embodiment, the amount of labeled sialic acid residues is quantified using a plate reader.

In one embodiment, the biomolecule of interest is a glycoprotein, a glycolipid, a glycophosphoinositol, an oligosaccharide, or a polysaccharide. In one embodiment, the glycoprotein is an antibody or an Fc-domain containing fragment thereof.

The invention is also directed to a kit for detecting sialylation of a biomolecule of interest in a sample comprising: (a) a solid support purification kit, a denaturing agent, a reagent suitable for removing a terminal sialic acid residue from a biomolecule of interest, or a labeling reagent suitable for detectably labeling free sialic acid residues, and (b) instructions describing how to use the one or more reagents to detect sialylation of the biomolecule of interest. In one embodiment, the kit comprises protein A-coated resins loaded in the solid support purification kit. In another embodiment, the solid support purification kit comprises 96-wells. In another embodiment, the denaturing agent is sodium 3-[(2-methyl-2-undecyl-1,3-dioxolan-4-yl)methoxy]-1-propanesulfonate. In another embodiment, the reagent for removing a terminal sialic acid residue from the biomolecule of interest is sialidase. In another embodiment, the labeling regent is malononitrile.

### BRIEF DESCRIPTION OF THE DRAWINGS/FIGURES

FIG. 1: Schematic of high-throughput method for quantifying glycoprotein sialylation. The high-throughput method consists of four steps. First, the samples are purified using a high-throughput purification system that can simultaneously purify 96 samples within ˜30 minutes. Here, the chemicals (◯), host cell proteins, and other biomolecules ( [symbol] ) are removed to obtain purified product ( [symbol] ). Second, Rapigest SF is added to denature the purified protein (30 min). Third, the sialic acid () is cleaved by sialidase (5 min). Fourth, the released sialic acid is derivatized by malononitrile, and the fluorescence of the product (★) is used to calculate the sialic acid concentration. The concentration of sialic acid is then divided by the purified protein concentration to obtain the sialic acid content.

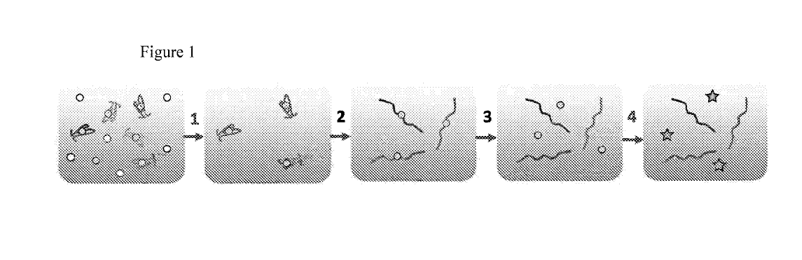

FIG. 2. The high-throughput method (HTM) is specific and sensitive. (a) Fluorescence measured by HTM was specific to sialic acid released from the glycoprotein of interest. Sample 1 was a positive control, in which elution and neutralization buffer mixture was analyzed by HTM, but 200 μM sialic acid standard, instead of sialidase, was added in the third step to mimic the release of the sialic acid from the protein. Sample 2 was similar to sample 1, but sialic acid standard was not added. The presence of fluorescence in sample 1, but not 2, showed that the fluorescence was due to the sialic acid. Sample 3 and 4 contained 1 g/L recombinant protein P1 in the same elution and neutralization buffer mixture, but sialidase was not added to sample 4 in the third step. Fluorescence was observed only in sample 3, confirming that the fluorescence was due to sialic acid released from P1. (b) Standard curve of HTM was linear from 0 to 200 μM sialic acid. Here sialic acid standard was added at different concentrations to the buffer mixture in the third step of HTM (similar to sample 1 of FIG. 2a). The quantitation limit of HTM calculated from this standard curve was 1 μM sialic acid. Error bars in (a) and (b) (not seen) were SD (n=3).

FIG. 3. The high-throughput method (HTM) is accurate and precise. (a) HTM was accurate relative to HPLC method. 45 purified P1 samples were analyzed by both HTM and HPLC. Each of the HTM measurement was repeated independently three times, while not all of the HPLC measurements were repeated due to the large number of samples and low throughput of the HPLC. Solid line corresponds to y=x, and dashed lines correspond to y=(1±0.08) x. Error bars were SD (n=3). (b) Differences between HTM and HPLC data were no more than 8%. (c) Distribution of coefficient of variation (CV) of each of the 45 HTM measurements showed that all of the data points had CV≦6%, indicating that the HTM was precise.

FIG. 4. The high-throughput method (HTM) has no significant well-to-well and day-to-day variability. (a) 48 replicates of 1 g/L P1 were analyzed in a 96-well-plate. The sialic acid content was 10.6±0.4 mol sialic acid/mol P1. Solid line corresponds to y=10.6 mol sialic acid/mol P1, and dashed lines correspond to y=10.6±0.5 mol sialic acid/mol P1. (b) Sialic acid content of 1 g/L P1 was measured by HTM on four different days. Confidence intervals of all the four measurements based on student's t-distribution (α=0.05) overlapped with each other, indicating that there was no statistically significant day-to-day variability.

### DETAILED DESCRIPTION OF THE INVENTION

As used herein, the terms “about” and “approximately”, as applied to one or more particular cell culture conditions, refer to a range of values that are similar to the stated reference value for that culture condition or conditions. In certain embodiments, the term “about” refers to a range of values that fall within 25, 20, 19, 18, 17, 16, 15, 14, 13, 12, 11, 10, 9, 8, 7, 6, 5, 4, 3, 2, 1 percent or less of the stated reference value for that culture condition or conditions.

In general, the methods provided herein use chemical and enzymatic reactions to detect and/or quantify the sialylation of glycosylated biomolecule of interest. The methods provided herein include four steps: purification of biomolecule of interest, denaturation of the biomolecule of interest, cleavage of terminal sialic acid from the biomolecule of interest, and derivatization of sialic acid using labeling to produce a detectably labeled sialic acid residue. The detectably labeled sialic acid can optionally be quantified. The term “sialic acid”, as used herein, is a generic term for the N- or O-substituted derivatives of neuraminic acid, a nine-carbon monosaccharide. The amino group of neuraminic acid typically bears either an acetyl or a glycolyl group in a sialic acid. The hydroxyl substituents present on the sialic acid may be modified by acetylation, methylation, sulfation, and phosphorylation. The predominant sialic acid is N-acetylneuraminic acid (Neu5Ac). Sialic acids impart a negative charge to glycans, because the carboxyl group tends to dissociate a proton at physiological pH.

In one embodiment, the detection of sialylation is an automated process. In another embodiment, at least one step of the claimed method is performed using robotics.

The methods of the invention are particularly useful in that they can be used to analyze multiple samples in a high-throughput format. In one embodiment, the samples to be analyzed are is located in a solid support or vessel. “Solid support”, “support”, and “vessel” are used interchangeably and refer to a material or group of materials having a rigid or semi-rigid surface or surfaces. In many embodiments, at least one surface of the solid support will be substantially flat, although in some embodiments it may be desirable to physically separate regions for different compounds. In one embodiment, the multi-well vessel comprises up to 96 wells. In another embodiment, the multi-well vessel comprises greater than 96 wells, for example 384 or 1536 wells.

In one embodiment, methods of detecting and optionally quantitating sialylation of one or more biomolecules of interest in a sample comprise purifying the biomolecule of interest. In one embodiment, the biomolecule of interest is purified by removal of host cell proteins, peptides, lipids, chemical components of culture media, and other impurities from the sample. Counter to previous detection methods (see, US Pub. No. 2011/0086362), it is demonstrated in the present application that removal of host cell proteins and other impurities is necessary to eliminate interference caused by these contaminants and provides specificity for detection of sialylation for the biomolecule of interest. The sample can be purified using an agent that binds the biomolecule of interest, including but not limited to antibodies, lectins, antigens, and receptors specific to the biomolecule of interest. In one embodiment, affinity chromatography is the separation technique used to purify a biomolecule of interest. In one embodiment, the affinity ligand is protein A or protein G. In certain embodiments, protein A is immobilized onto a solid support and the biomolecule of interest is bound to the immobilized protein A, thereby removing other proteins, peptides, lipids, chemicals, and other impurities in the sample. The purified protein is transferred into a new vessel for denaturation.

In other embodiments, a glycosylated polypeptide of interest is directly purified with an affinity ligand. In certain embodiments, the glycosylated polypeptide of interest is an antibody and the cognate antigen or a lectin is used to purify the polypeptide from the sample. When a lectin is used, proteins and other molecules that do not bind to the lectin are washed away and then specifically bound glycoproteins can be eluted by adding a high concentration of a sugar that competes with the bound glycoproteins at the lectin binding site. In one embodiment, at least two purification steps are performed. In certain embodiments, purification with a lectin is performed after an affinity chromatography separation technique.

The sample can be denatured by contact with a denaturing agent. “Denaturation” as used herein means a process in which proteins reduce or lose their tertiary and/or secondary structures by application of compound(s), and/or by external stress such as, for example, heat. The denaturing agents help to expose the sialic acid of the biomolecule of interest, making them more susceptible to enzymatic cleavage. In one embodiment, the denaturing agent is a surfactant. Any surfactant that does not interfere with sialidase activity or malononitrile derivatization can be used for protein denaturation. Denaturing surfactants are known in the art and include, but are not limited to, sodium 3-[(2-methyl-2-undecyl-1,3-dioxolan-4-yl)methoxy]-1-propanesulfonate, glycolic acid ethoxylate lauryl ether, N,N-dimethyl-N-[3-(sulfooxy)propyl]-1-nonanaminium hydroxide inner salt, poly(ethylene glycol) 4-nonylphenyl 3-sulfopropyl ether potassium salt, and sodium dodecylbenzenesulfonate.

The denaturing agent is allowed to react with the sample for a time suitable to partially unfold the polypeptide. Suitable incubation times can be, for example, about 5 to 60 minutes. In one embodiment, the sample is incubated for 30 minutes. Denaturation is generally terminated by heat inactivation. In certain embodiments, the denatured solution is heated to about 60° C. and allowed to cool to room temperature. Suitable concentrations, incubation times, and incubation temperatures can be determined by one or ordinary skill in the art using routine optimization methods.

The denatured sample is contacted with an agent that is capable of removing terminal sialic acid residues from the biomolecule of interest, generating free sialic acid residues. Terminal sialic acid residues can be removed using any suitable method that specifically removes the terminal sialic acid without removing other sugar residues that may react with the labeling agent. In this way, the released or free sialic acid residue is capable of being detectably labeled with the labeling agent. The sialic acid can be released using chemical or enzymatic means. In some embodiments, an enzyme is used. In some embodiments the enzyme sialidase is used. A “sialidase” as used herein refers to an enzyme that hydrolyses alpha-(2->3)-, alpha-(2->6)-, alpha-(2->8)-glycosidic linkages of terminal sialic residues in oligosaccharides, glycoproteins, and glycolipids. Enzymes for removing terminal sugar residues are commercially available and can be used according to the manufacturer's instructions. In some embodiments, the terminal sialic acid residues are released by incubating the denatured sample/agent mixture at 37° C. In another embodiment, the terminal sialic acid residues are released using mild acid hydrolysis.

The free sialic acid residues are detectably labeled with a labeling agent, and the labeled free sialic acid residues are detected. In some embodiments, sialylation of the one or more glycosylated molecules is quantified. Free sialic acid residues can be detectably labeled using any agent that is capable of detectably labeling a free sugar residue. In some embodiments, any excess or unreacted labeling agent is not be detectable, e.g., is not fluorescent. In some embodiments, if excess or unreacted labeling reagent is fluorescent or otherwise detectable, it is detectable at different excitation or emission wavelength than the labeled free sugar residue. In some embodiments, the labeling agent is capable of detectably labeling sialic acid residues. In some embodiments, the labeling reagent is malononitrile Suitable concentrations of labeling agent can be, for example, about 0.5 to about 15 g/L. In some embodiments, suitable concentrations of labeling reagent can be, for example, at least 7 g/L. The labeling agent can be allowed to react with the sample for a time suitable to detectably label free sialic acid residues. Suitable incubation times can be, for example, about 30 seconds to about 30 minutes, depending on the labeling agent used. In some embodiments, suitable incubation times can be, for example, between 1 and 5 minutes. The labeling agent can be allowed to react with freed sialic acid residues at a temperature suitable to detectably label free sialic acid residues. Suitable incubation temperatures can be, for example, between about 4 and about 100° C. Suitable concentrations, incubation times, and incubation temperatures can be determined by one or ordinary skill in the art using routine optimization methods.

The label can be any label that is detected, or is capable of being detected. Examples of suitable labels include, e.g., chromogenic label, a radiolabel, a fluorescent label, a luminescent label, and a biotinylated label. Thus, the label can be, e.g., colored lectins, fluorescent lectins, biotin-labeled lectins, fluorescent labels, fluorescent antibodies, biotin-labeled antibodies, and enzyme-labeled antibodies. In one embodiments, the label is a chromogenic label. The term “chromogenic binding agent” includes all agents that bind to proteins or saccharides and which have a distinct color or otherwise detectable marker, such that following binding to a saccharide, the saccharide acquires the color or other marker. In addition to chemical structures having intrinsic, readily-observable colors in the visible range, other markers used include fluorescent groups, biotin tags, enzymes (that may be used in a reaction that results in the formation of a colored product), magnetic and isotopic markers, and so on. The foregoing list of detectable markers is for illustrative purposes only, and is in no way intended to be limiting or exhaustive. In a similar vein, the term “color” as used herein (e.g. in the context of step (e) of the above described method) also includes any detectable marker.

The label may be attached to the agent using methods known in the art. Labels include any detectable group attached to the glycoprotein, or detection agent that does not interfere with its function. Labels may be enzymes, such as peroxidase, luciferase, and phosphatase. In principle, also enzymes such as glucose oxidase and β-galactosidase could be used. It must then be taken into account that the saccharide may be modified if it contains the monosaccharide units that react with such enzymes. Further labels that may be used include fluorescent labels, such as Fluorescein, Texas Red, Lucifer Yellow, Rhodamine, Nile-red, tetramethyl-rhodamine-5-isothiocyanate, 1,6-diphenyl-1,3,5-hexatriene, cis-Parinaric acid, Phycoerythrin, Allophycocyanin, 4′,6-diamidino-2-phenylindole (DAPI), Hoechst 33258, 2-aminobenzamide, and the like. Further labels include electron dense metals, such as gold, ligands, haptens, such as biotin, radioactive labels.

The agent can additionally be detected using enzymatic labels. The detection of enzymatic labels is well known in the art. Examples include, e.g., ELISA and other techniques where enzymatic detection is routinely used.

In some embodiments, the label is detected using fluorescent labels. Fluorescent labels require an excitation at a certain wavelength and detection at a different wavelength. Fluorescent labels and methods for fluorescent detection are well known in the art.

The labeling agent may itself contain a carbohydrate moiety and/or protein. Coupling labels to proteins and sugars are techniques well known in the art. For instance, commercial kits for labeling saccharides with fluorescent or radioactive labels are available from Oxford Glycosystems, Abingdon, UK, and ProZyme, San Leandro, Calif. USA).

Coupling is usually carried out by using functional groups, such as hydroxyl, aldehyde, keto, amino, sulfhydryl, carboxylic acid, or the like groups. In addition, bifunctional cross-linkers that react with the label on one side and with the protein or saccharide on the other may be employed. The use of cross-linkers may be advantageous in order to avoid loss of function of the protein or saccharide.

As discussed above, the detectably-labeled free sialic acid residues can be detected using methods compatible with the chosen labeling agent. Colorimetric labeling agents can be detected using a spectrophotometer set at the appropriate wavelength for the colorimetric product. Fluorogenic labeling agents or fluorescent products thereof can be detected using a fluorometer.

In some embodiments of the methods provided herein, sialylation of the biomolecule of interest is quantified. In some embodiments, concentration of free sialic acid residues is determined by measuring fluorescence intensity of the detectably labeled sialic acid residues. The intensity of the signal of the derivatized sialic acid can be used to estimate the concentration of sialic acid released from glycoproteins using the Beer-Lambert law. The concentration can then be divided by the concentration of glycosylated biomolecule of interest providing the mole ratio of terminal monosaccharides to glycosylated biomolecule of interest. The concentration of glycosylated biomolecule of interest in the sample can be determined using standard techniques, such as enzyme-linked immunosorbant assay (ELISA), HPLC, or absorbance at 280 nm.

Suitable glycosylated polypeptides of interest include, for example, glycoproteins, glycolipids, oligosaccharides, and polysaccharides. As used herein, the term “glycoprotein” refers to a protein that contains a peptide backbone covalently linked to one or more sugar moieties (i.e., glycans). As is understood by those skilled in the art, the peptide backbone typically comprises a linear chain of amino acid residues. The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. In certain embodiments, sugar moieties may include sulfate and/or phosphate groups. Alternatively or additionally, sugar moieties may include acetyl, glycolyl, propyl or other alkyl modifications. In certain embodiments, glycoproteins contain O-linked sugar moieties; in certain embodiments, glycoproteins contain N-linked sugar moieties. In certain embodiments, methods disclosed herein comprise a step of analyzing any or all of cell surface glycoproteins, liberated fragments (e.g., glycopeptides) of cell surface glycoproteins, cell surface glycans attached to cell surface glycoproteins, peptide backbones of cell surface glycoproteins, fragments of such glycoproteins, glycans and/or peptide backbones, and combinations thereof.

The term “glycolipid” as used herein refers to a lipid that contains one or more covalently linked sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may be comprised of one or more branched chains. In certain embodiments, sugar moieties may include sulfate and/or phosphate groups. In certain embodiments, glycolipids contain C-linked sugar moieties; in certain embodiments, glycolipids contain N′-linked sugar moieties. Suitable glycolipids include, for example, glycophosphatidylinositol (GPI) and gangliosides. As used herein, an oligosaccharide comprises a short chain of carbohydrate structures (or sugar residues) having three to ten repeating units. Suitable oligosaccharides include, for example, sialyl Lewis a (sLe^{a}), sialyl Tn (sTn), sialyl-Lewis x (sLe^{x}), 6-sulpho-sLe^{x}, and sialylated glycans cleaved from glycoproteins or glycolipid by endoglycosidase. As used herein, a polysaccharide comprises a chain of carbohydrate structures having more than ten repeating units. Suitable polysaccharides include, for example, sialic acid-containing meningococcal serogroup B and C polysaccharides, polysialic acid (PSA). In certain embodiments, the biomolecule of interest is an antibody or an Fc domain-containing fragment thereof.

Samples suitable for use in the methods and with the kits provided herein can contain other components in addition to the one or more glycosylated molecules of interest. For example, the sample can contain free sialic acid residues, other free sugar molecules, proteins, cells, nucleic acids, and the like. Samples suitable for use in the methods and with the kits provided herein include any fluid or suspension suspected of containing the one or more glycosylated polypeptides of interest. In some embodiments, the sample is a biological sample. The one or more glycosylated polypeptides present in the sample can be recombinantly produced, for example by a recombinant host cell.

As described herein, recombinant host cells can be any suitable cell that has been altered to generate the one or more glycosylated polypeptides of interest, for example, by expressing an exogenous gene or nucleic acid that codes for the one or more glycosylated polypeptides of interest such that the molecule is expressed in glycosylated form. In some embodiments, the recombinant host cell is a fungal cell, insect cell, or mammalian cell into which one or more exogenous nucleic acid sequences have been added. Such nucleic acid sequences include constructs that are capable of expressing, for example, a glycosylated polypeptide of interest. The exogenous nucleic acid sequence can be introduced into the host cell by, for example, transfection using suitable transfection methods known in the art, or can be introduced by infection using a suitable viral vector, such that the glycosylated polypeptide of interest encoded by the exogenous nucleic acid is expressed in the host cell.

“Culture”, “cell culture” and “mammalian cell culture” as used herein refer to a mammalian cell population that is suspended in a cell culture medium under conditions suitable to survival and/or growth of the cell population. These terms as used herein may refer to the combination comprising the mammalian cell population and the medium in which the population is suspended.

Any mammalian cell or cell type susceptible to cell culture, and to expression of polypeptides, may be utilized in accordance with the present invention. Non-limiting examples of mammalian cells that may be used in accordance with the present invention include BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells +/−DHFR (CHO, Urlaub and Chasin, Proc. Natl. Acad. Sci. USA, 77:4216 (1980)); mouse sertoli cells (TM4, Mather, Diol, Reprod., 23:243-251 (1980)); monkey kidney cells (CV1 ATCC CCL 70); African green monkey kidney cells ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Rep G2, HB 8065); mouse mammary tumor (MMT 060562, ATCC CCL51); TR1 cells (Mather et al., Annals N.Y. Acad. Sci., 383:44-68 (1982)); MRC 5 cells; FS4 cells; and a human hepatoma line (Hep G2). In a particularly preferred embodiment, the present invention is used in the culturing of and expression of polypeptides and proteins from CHO cell lines.

Additionally, any number of commercially and non-commercially available hybridoma cell lines that express polypeptides or proteins may be utilized in accordance with the present invention. One skilled in the art will appreciate that hybridoma cell lines might have different nutrition requirements and/or might require different culture conditions for optimal growth and polypeptide or protein expression, and will be able to modify conditions as needed.

As noted above, in many instances the cells will be selected or engineered to produce high levels of protein or polypeptide. Often, cells are genetically engineered to produce high levels of protein, for example by introduction of a gene encoding the protein or polypeptide of interest and/or by introduction of control elements that regulate expression of the gene (whether endogenous or introduced) encoding the polypeptide of interest.

Suitable fungi include, for example, Saccharomyces cerevisiae or Pichia pastoris.Suitable insect cells include, for example, SF9 cells. In some embodiments, the bioprocess fluid can contain an endogenous product molecule, for example, clotting factors and immunoglobulin can be isolated from blood or fractions thereof.

The sample can be derived from any biological source, such as a physiological fluids (e.g., blood, saliva, sputum, plasma, serum, ocular lens fluid, cerebrospinal fluid, sweat, urine, milk, ascites fluid, synovial fluid, peritoneal fluid, amniotic fluid, cell membrane suspensions, and the like). In addition, the sample can be biopsy material. The sample can be obtained from a human, primate, animal, avian or other suitable source. The sample can also be plant material or cells. The sample can be from prokaryote that has been engineered to produce glycosylated molecules. Suitable plant material can be obtained, for example from tobacco. Suitable plant cells can be, for example tobacco BY2 cells (GT6 cells). Suitable prokaryotes include, for example E. coli.

Samples from tissue or cellular material can be processed into a fluid form suitable for use in the methods provided herein. Suitable processing are known in the art and include, but are not limited to homogenization, sonication, cavitation and the like. The processing can include use of detergents, saponification agents and enzymes to digest or remove molecules such as nucleic acids. The processing can include centrifugation or filtration to remove non-solubilized material.

In some embodiments, the sample is a bioprocess fluid that contains the one or more glycosylated polypeptides of interest. For example, the bioprocess fluid can be conditioned culture medium, or milk, blood, plasma, plasma fractions, urine, ascites fluid and the like. In other embodiments, the bioprocess fluid comprises cell homogenates, cell extracts, tissue homogenates, tissue extracts, and the like. The sample can be processed as described herein.

### Kits

The invention further relates to a method of detection/quantification of terminal sialylation can be commercialized as a kit. In certain embodiments, the kit is designed for use in a high-throughput format. The kit can include one or more reagents suitable for detecting and optionally quantifying sialylation of glycosylated biomolecules of interest as described herein. The provided reagents can be one or more of: a high-throughput purification kit, a denaturing reagent, a reagent suitable for removing a terminal sialic acid residue from a glycoprotein, and a labeling reagent suitable for detectably labeling free a sialic acid residue. The kits provided herein include instructions describing how to use the one or more reagents to detect sialylation of one or more glycosylated biomolecules of interest in a sample and optionally how to quantify sialylation of one or more glycosylated molecules in a sample. The kit can include one or more multi-well plates, such as 96-well-plates or PCR plates. The kits provided herein can include standard solutions for calibrating the fluorescence intensity for a given sugar residue of interest and labeling agent, and/or reagents to quantify the sialylation of glycoproteins.

In addition to detecting and/or quantifying sialylation of glycosylated biomolecules of interest, the methods and kits provided herein can be used for various applications, such as monitoring of sialylation during production of therapeutic glycoproteins, and optimization of glycoprotein sialylation by changing various culture condition and supplementation, and analyzing patient samples for alterations in sialylation of glycosylated biomolecules of interest.

The methods provided herein can be used to quantify other terminal sugar residues such as mannose, galactose, fucose, and N-acetylglucosamine using the appropriate reagent to remove the terminal sugar residue of interest. For example, terminal galactose residues can be removed using the enzyme α-galactosidase or β-galactosidase, terminal fucose residues can be removed using the enzyme α-fucosidase, and terminal N-acetylglucosamine residues can be removed using the enzyme β-N-acetylglucosaminidase. These enzymes are commercially available.

### EXAMPLES

### Method for High-Throughput Quantification of Sialic Acid

HTM consists of four steps. First, crude recombinant P1 samples harvested from cell cultures were purified using a protein A coated resin loaded into a 96-well plate. This purification took ˜20 minutes. Second, 30 μL of purified P1 samples were pipetted into a PCR plate, and mixed with 30 at, mixture of sodium phosphate buffer (100 mM, pH=6.0) and NaOH (5M) to increase the pH to 6.0. Afterwards, 15 μL of 0.5% (w/v) Rapigest SF (Waters) prepared in the same buffer was added to and mixed with the samples. These samples were then heated at 60° C. for 30 min, and briefly cooled to room temperature to stop the denaturation step. Third, 6 μL of diluted sialidase (0.015 U from Arthrobacter ureafaciens, Roche, diluted in 20 mM Tris-HCl, 25 mM NaCl, pH=7.0) was added to the mixture. It was then mixed, heated at 37° C. for 5 min, and briefly cooled to room temperature. Fourth, 111 μL sodium borate buffer (0.15M, pH=9.4) and 12 μL malononitrile (8 g/L) were added to the mixture. The mixture was heated at 80° C. for 5 min, and briefly cooled on ice bath to stop the reaction. An aliquot (175 of the mixture was then transferred from the PCR plate to a black 96-well plate for fluorescence measurement using a plate reader (Victor X3, Perkin Elmer) at λₑₓ=355 nm and λₑₘ=430 nm. The fluorescence intensity was used to calculate the sialic acid concentration. To obtain the sialic acid content, this concentration was divided by protein concentration measured using A₂₈₀. Fetuin and α1-acid glycoprotein used in our analyses were bought from Sigma.

### HPLC Sialic Acid Assay

Purified P1 samples were hydrolyzed by mild acid hydrolysis. The acid hydrolyzed samples were then analyzed by HPLC using an ion-exclusion column, isocratic mobile phase, under UV detection (M. G. Steiger et al. Microbial Cell Factories 10 (2011)).

### Detection of Sialylated Polypeptides of Interest

In the previous HTM, tetrabutylammonium borohydride (Bu₄NBH₄) was used to eliminate interference by chemical components of crude culture samples, such as glucose (L. R. A. Markely et al. Anal. Biochem. 407:128-133 (2010)), Nonetheless, significant interference by sialic acid released from other biomolecules than recombinant IFN-γ in culture samples was observed. In order to avoid the interference, a high-throughput protein purification was used to remove the chemicals, HCP, and other biomolecules. In this first step, 96 samples can simultaneously be purified and concentrated (one to a few mL each) within ˜30 minutes.

In the second step, the purified protein is denatured using a surfactant, sodium 3-[(2-methyl-2-undecyl-1,3-dioxolan-4-yl)methoxy]-1-propanesulfonate (Rapigest SF) at 60° C., pH ˜6.0 for 30 minutes. The denaturation step is required to obtain complete cleavage of sialic acid by sialidase (Table 1).

TABLE 1

| | Sialic acid content (mol sialic acid/mol protein) | | |
|---|---|---|---|
| Protein | HTM − Rapigest SF | HTM + Rapigest SF | HPLC |
| Fetuin | 9.0 ± 0.3 | 12.7 ± 0.3 | 11.3 ± 0.1 |
| α1-acid | 12.5 ± 1.4 | 15.6 ± 0.4 | 16.7 ± 0.1 |
| glycoprotein | | | |
| P1 | 8.4 ± 0.4 | 10.5 ± 0.4 | 12.0 ± 0.1 |
| P2 | 10.6 ± 0.6 | 16.4 ± 0.6 | 17.0 ± 0.0 |
| P3 | 0.4 ± 0.0 | 0.6 ± 0.0 | 1.0 ± 0.0 |

Table 1. Protein denaturation is required to ensure complete cleavage of sialic acid from glycoproteins. Sialic acid content of fetuin, α1-acid glycoprotein, three different types of recombinant proteins (P1, P2, and P3) are measured using HPLC, HTM with, and without denaturation by Rapigest SF. The sialic acid contents measured by HTM with denaturation were higher than those without denaturation, and were similar to HPLC data. Uncertainties indicate SD (n=3).

When this denaturation step was included, the sialic content measured by HTM was similar to an established mild acid hydrolysis HPLC method. When the denaturation step was not included, the HTM data were lower than the HPLC data. This observation indicated that some of the sialic acid was not accessible to sialidase, and the denaturation was required to expose the sialic acid to the sialidase. Moreover, the protein denaturation is required not only for analysis of P1, but also four other types of proteins, including fetuin, α1—acid glycoprotein, recombinant fusion protein P2, and monoclonal antibody P3.

In the third step, sialic acid bound to the protein is released by sialidase at 37° C., pH˜6.0 for 5 minutes. This step is followed by malononitrile derivatization at 80° C., pH˜9.4 for 5 minutes. In the derivatization, the product is fluorescent, but the sialic acid and malononitrile are not fluorescent (L. R. A. Markely et al. Anal. Biochem. 407:128-133 (2010); S. Honda et al. Anal. Biochem. 160:455-461 (1987); and K. Li, J. Chromatography-Biomedical Appl. 579:209-213 (1992)). Thus, removal of excess malononitrile is not required, and the fluorescent intensity can be directly used to calculate the sialic acid concentration in the samples. The sialic acid concentration is then divided by concentration of purified protein to obtain the sialic acid content.

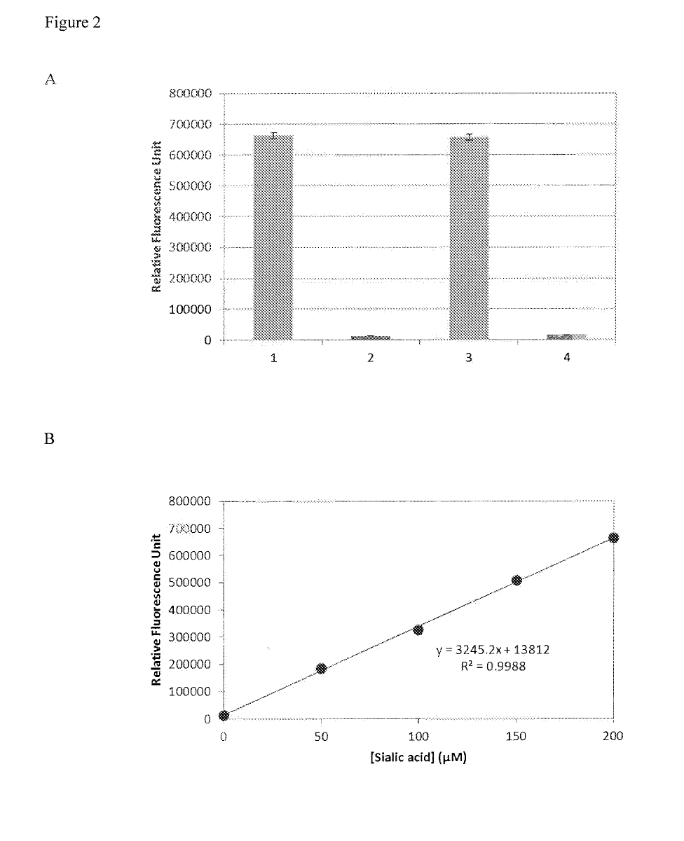

The current HTM was specific to sialic acid of glycoproteins (FIG. 2a). Sample 1 was a buffer used in the high-throughput protein purification. It was analyzed by HTM as described above, except that sialic acid standard, instead of sialidase, was added in the third step. This addition was done to mimic the release of sialic acid from glycoproteins. Sample 2 was similar to sample 1, but the sialic acid standard was not added in the third step. The absence of fluorescence of sample 2 indicated that the fluorescence of sample 1 was specifically due to the sialic acid standard. Sample 3 contained a recombinant protein, P1, and was analyzed by HTM as described above. Sample 4 was identical to sample 3, but sialidase was not added in the third step. Fluorescence was observed in sample 3, but not sample 4, showing that the fluorescence was specifically due to sialic acid released from P1.

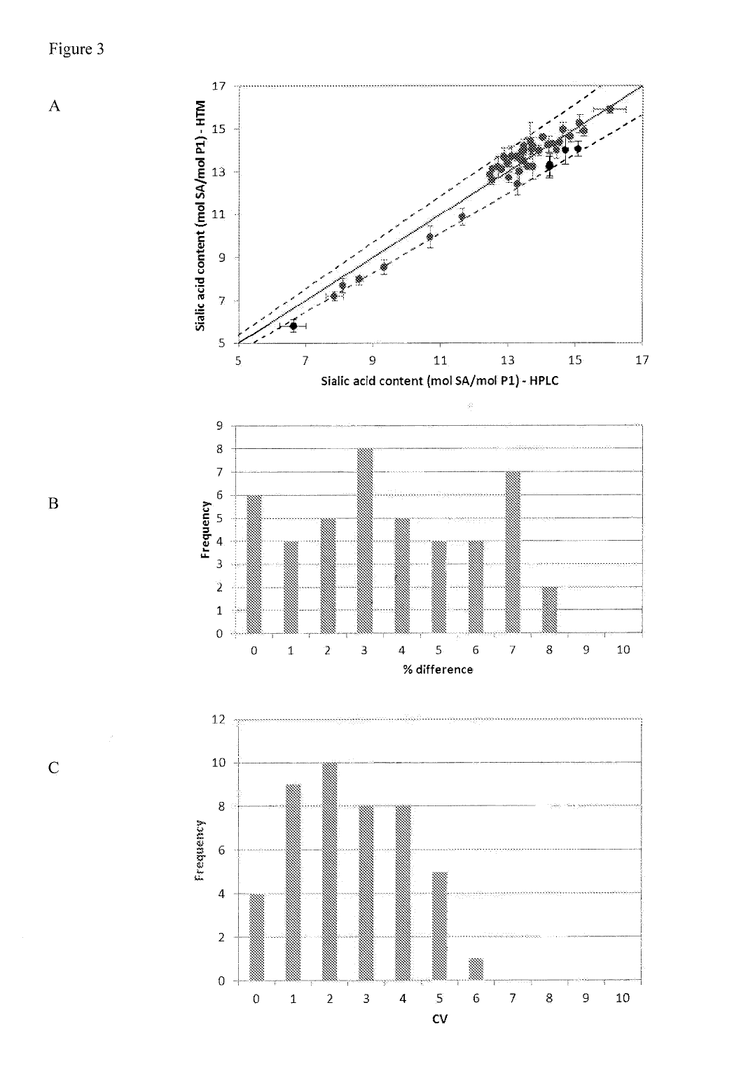

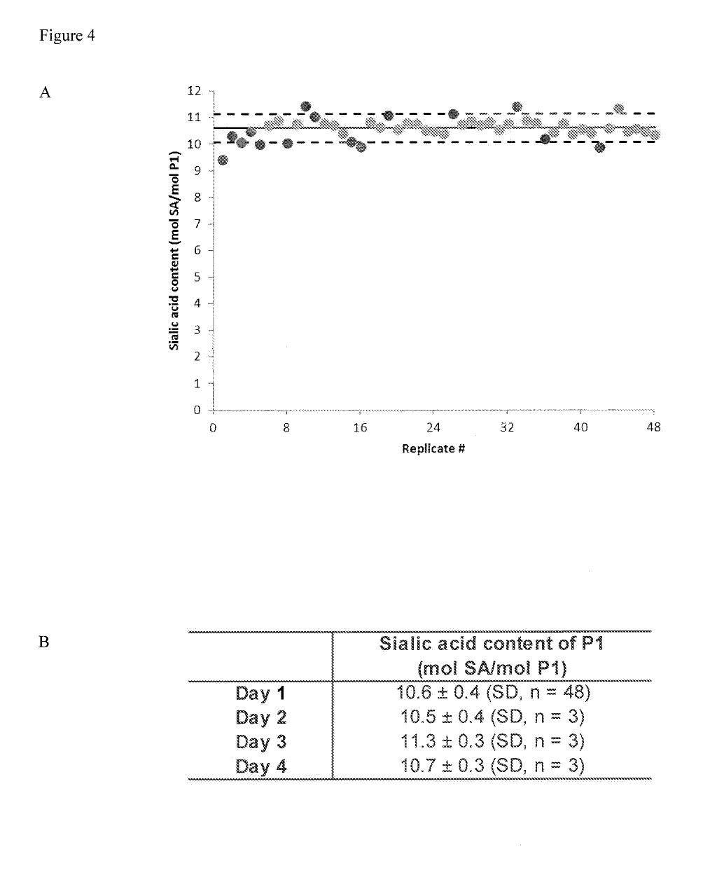

The HTM was also sensitive, accurate, and precise. The standard curve was linear from 0 to 100 μM sialic acid (FIG. 2b). The quantitation limit, ten times standard deviation of the blank of the calibration curve, was 1 μM sialic acid. This quantitation limit is equivalent to 30 pmol sialic acid (30 μL of sample was required by the HTM). Moreover, comparison between HTM and HPLC data showed that the HTM was accurate (FIG. 3a and Table 2). Here 45 purified P1 samples were analyzed by both HTM and HPLC. These samples were harvested from recombinant cell cultures. The sialic acid contents of the 45 P1 samples measured by HTM and HPLC were similar; the differences among the data were ≦8% (FIG. 3b). Furthermore, each of the 45 HTM measurements was repeated independently three times, and the coefficient of variation (CV) was calculated for each measurement. All of the data points have CV≦6%, showing that the HTM was precise (FIG. 3c). Similarly, the variability of the HTM observed among 48 replicates in a 96-well-plate (FIG. 4a) and four runs performed on four different days (FIG. 4b) was insignificant.

Overall, this HTM is specific, sensitive, accurate, and precise. In order to achieve the accuracy, protein denaturation with Rapigest SF was required. Moreover, this HTM can analyze at least 80 crude culture supernatants within 70 minutes. Compared to the previous methods, the current HTM has several advantages. First, it is free from interference by any sialylated biomolecule in crude culture supernatants. Thus, it can be used to specifically measure the sialylation of the product (mol sialic acid/mol product) instead of overall sialylation (mg sialic acid/g total protein) measured by the previous HTM. Second, it is more convenient to use than the previous HTM because the current HIM uses only aqueous buffers and does not use tetrahydrofuran (THF). Consequently, the risk of protein aggregation caused by mixing organic and aqueous solutions can be avoided. In addition, removing the THF make the current. HTM easily adaptable for robotic automation. With these modifications, the current HTM can readily be used to accelerate and improve cell line and bioprocess development for producing therapeutic proteins with consistent and optimum quality.

TABLE 2

| Sample | HTM | HPLC |
|---|---|---|
| 1 | 14.3 ± 0.1 | 14.3 ± 0.0 |
| 2 | 14.2 ± 0.4 | 13.7 |
| 3 | 14.0 ± 0.4 | 14.4 |
| 4 | 13.3 ± 0.5 | 14.2 |
| 5 | 13.2 ± 0.5 | 14.2 |
| 6 | 13.2 ± 0.6 | 13.7 |
| 7 | 13.0 ± 0.6 | 13.3 |
| 8 | 13.7 ± 0.5 | 13.1 |
| 9 | 13.2 ± 0.5 | 12.8 ± 0.1 |
| 10 | 12.4 ± 0.5 | 13.3 |
| 11 | 14.4 ± 0.8 | 13.7 |
| 12 | 14.2 ± 0.3 | 13.5 |
| 13 | 13.5 ± 0.3 | 13.4 |
| 14 | 12.7 ± 0.3 | 13.0 |
| 15 | 14.6 ± 0.3 | 14.9 |
| 16 | 14.1 ± 0.4 | 15.1 |
| 17 | 13.2 ± 0.1 | 13.6 |
| 18 | 12.6 ± 0.2 | 12.5 |
| 19 | 14.3 ± 0.4 | 14.2 |
| 20 | 14.0 ± 0.3 | 13.7 |
| 21 | 13.7 ± 0.2 | 13.3 |
| 22 | 13.1 ± 0.0 | 12.5 |
| 23 | 15.2 ± 0.4 | 15.1 |
| 24 | 14.9 ± 0.3 | 14.6 |
| 25 | 14.6 ± 0.0 | 14.0 |
| 26 | 14.0 ± 0.1 | 13.5 |
| 27 | 13.9 ± 0.1 | 13.4 |
| 28 | 13.6 ± 0.4 | 12.9 |
| 29 | 13.4 ± 0.0 | 13.0 |
| 30 | 12.9 ± 0.1 | 12.5 |
| 31 | 14.0 ± 0.7 | 14.7 |
| 32 | 14.2 ± 0.2 | 14.2 |
| 33 | 13.5 ± 0.1 | 13.4 |
| 34 | 13.1 ± 0.0 | 12.8 |
| 35 | 15.9 ± 0.2 | 16.6 ± 0.5 |
| 36 | 14.9 ± 0.2 | 15.2 |
| 37 | 14.4 ± 0.2 | 14.5 |
| 38 | 14.0 ± 0.3 | 13.9 |
| 39 | 5.8 ± 0.3 | 6.2 ± 0.4 |
| 40 | 7.2 ± 0.2 | 7.8 ± 0.3 |
| 41 | 7.7 ± 0.3 | 8.1 |
| 42 | 8.0 ± 0.3 | 8.7 ± 0.1 |
| 43 | 8.5 ± 0.3 | 9.3 |
| 44 | 9.9 ± 0.5 | 10.7 |
| 45 | 10.9 ± 0.4 | 11.7 |

Table 2: High-throughput method (HTM) was accurate. 45 purified P1 samples were analyzed by both HTM and HPLC. Each of the HTM measurement was repeated independently three times, while not all of the HPLC measurements were repeated three times due to the large number of samples and low throughput of the HPLC method. The difference between HTM and HPLC data were no more than 8%. Uncertainties correspond to SD (n=3).

All publications and patents mentioned in the above specification are herein incorporated by reference. Various modifications and variations of the described method and system of the invention will be apparent to those in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those in the relevant fields are intended to be within the scope of the following claims.

What is claimed is:

1. A method of detecting sialylation of a biomolecule of interest in a sample comprising:
(a) purifying the biomolecule of interest by contacting the sample with an agent that binds the biomolecule of interest;
(b) denaturing the biomolecule of interest by incubating the sample with a surfactant;
(c) contacting the denatured sample with an agent capable of removing terminal sialic acid residues from the biomolecule of interest, thereby generating free sialic acid residues;
(d) labeling the free sialic acid residues with a detectable label;
(e) detecting the labeled sialic acid residues, thereby detecting sialylation of the biomolecule of interest; whereby said method allows for the detection of sialylation of the biomolecule of interest without interference from host cell proteins or impurities.

2. The method of claim 1, further comprising quantifying the level of sialylation of the biomolecule of interest.

3. The method of claim 1 or 2, wherein the sample comprises a cell culture supernatant.

4. The method of claim 1 or 2, wherein the sample comprises a clinical sample.

5. The method of any of claims 1-4, wherein the sample is located in a multi-well vessel.

6. The method of claim 5, wherein said multi-well vessel comprises up to 96 wells.

7. The method of claim 5, wherein said multi-well vessel comprises greater than 96 wells.

8. The method of claim 5, wherein said multi-well vessel comprises 384 wells.

9. The method of claim 5, wherein said multi-well vessel comprises 1536 wells.

10. The method of any of claims 1-9, wherein the agent that binds the biomolecule of interest is an antibody, lectin, antigen, or receptor that specifically binds the biomolecule of interest.

11. The method of any of claims 1-9, wherein the agent that binds the biomolecule of interest is protein A.

12. The method of any of claims 1-11, wherein the surfactant is sodium 3-[(2-methyl-2-undecyl-1,3-dioxolan-4-yl)methoxy]-1-propanesulfonate.

13. The method of any of claims 1-12, wherein the denaturation occurs between about 50° C. and about 80° C.

14. The method of claim 13, wherein the denaturation occurs at between about 60° C. and about 70° C.

15. The method of claim 14, wherein the denaturation occurs at about 60° C.

16. The method of any of claims 1-15, wherein the terminal sialic acid residues are removed by contacting the denatured sample with an enzyme.

17. The method of claim 16, wherein the enzyme is sialidase.

18. The method of any of claims 1-17, wherein the labeling agent is malonitrile.

19. The method of any of claims 1-18, wherein the labeling is performed at a temperature of between about 60° C. and about 100° C.

20. The method of claim 19, wherein the labeling is performed at 80° C.

21. The method of any of claims 1-20, wherein the free sialic acid is detected using a spectrophotometer or a fluorometer.

22. The method of any of claims 1-21, wherein the amount of labeled sialic acid residues is quantified using a plate reader.

23. The method of any of claims 1-22, wherein the biomolecule of interest is selected from the group consisting of: glycoproteins, glycolipids, glycophosphoinositols, oligosaccharides, and polysaccharides.

24. The method of claim 23, wherein the glycoprotein is an antibody or an Fc-domain containing fragment thereof.

25. A kit for detecting sialylation of a biomolecule of interest in a sample comprising: (a) a solid support purification kit, a denaturing agent, a reagent suitable for removing a terminal sialic acid residue from a glycoprotein, or a labeling reagent suitable for detectably labeling free sialic acid residues, and (b) instructions describing how to use the one or more reagents to detect sialylation of the biomolecule of interest.

26. The kit of claim 25, wherein the kit comprises protein A-coated resins loaded in the solid support purification kit.

27. The kit of claim 25 or 26, wherein the solid support purification kit comprises 96 wells.

28. The kit of any of claims 25-27, wherein the denaturing agent is sodium 3-[(2-methyl-2-undecyl-1,3-dioxolan-4-yl)methoxy]-1-propanesulfonate.

29. The kit of any of claims 25-28, wherein the reagent for removing a terminal sialic acid residue from a glycoprotein is sialidase.

30. The kit of any of claims 25-29, wherein the labeling regent is malononitrile.
